(19) United States
(12) Patent Application Publication
(10) Pub. No.: US 2009/0203670 A1
(43) Pub. Date: Aug. 13, 2009

(54) Combination Antidepressants Wafer

(75) Inventors: Hans-Rainer Hoffmann, Neuwied (DE); Reto Brandli, San Fransisco, CA (US); Frank Theobald, Wehr (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andernach (DE)

(21) Appl. No.: 12/308,194

(22) PCT Filed: Jun. 4, 2007
(86) PCT No.: PCT/EP2007/004936

§ 371 Date: Dec. 9, 2008

(30) Foreign Application Priority Data
Jun. 16, 2006 (DE) 102006027792.9

(51) Int. Cl. A61K 31/55; A61K 31/5375; A61K 31/497; A61K 31/519

(52) U.S. Cl. 514/214.02; 514/221; 514/239.2; 514/252.18; 514/259.41

(57) ABSTRACT

A sheet-like pharmaceutical preparation (dosage form) that quickly disintegrates on contact with moisture, based on hydrophilic polymers for the release of at least one active agent. The dosage form contains an active agent combination for treating depression, and at least one active agent selected from the antidepressant group of drugs. In addition, the use of such a combination of active agents for the production of an orally administrable pharmaceutical product for the treatment of depression, and the therapeutic treatment of depression by oral administration of one of the named pharmaceutical preparations, is provided.

### CROSS-REFERENCE TO RELATED APPLICATIONS

This application is a National Stage application of International Application No. PCT/EP2007/004936, filed on Jun. 4, 2007, which claims priority of German application number 10 2006 027 792.9, filed on Jun. 16, 2006, both of which are incorporated herein by reference in their entireties.

### BACKGROUND OF THE INVENTION

1. Field of the Invention

The present invention relates to a sheet-like pharmaceutical preparation that quickly disintegrates on contact with moisture, based on hydrophilic polymers for the release of at least one active agent in a body orifice or body cavity, wherein the dosage form contains an active agent combination for treating depression, and wherein at least one of the active agents is selected from the antidepressant group of drugs.

The invention further relates to the use of such a combination of active agents for the production of an orally administrable pharmaceutical preparation for the treatment of depression, and to the therapeutic treatment of depression by means of oral administration of one of the named pharmaceutical preparations.

2. Description of the Prior Art

In Germany, about 5% of the population suffer from depression requiring treatment. It is particularly striking that according to WHO data, women are afflicted about 2-3 times more often than men.

Depressions are defined in the literature as illnesses that are accompanied by dejection and additional physical and psychic disorders and which are characterized by the following essential symptoms:
depressed mood, lack of drive, fatigue;
low appetite and loss of weight;
sleep disorders;
restlessness, reticence, concentration difficulties, slow thinking and slow actions, indecisiveness;
risk of suicide.

Because of their wide-spread occurrence and particularly because of the risk of suicide, depressions constitute a serious, life-threatening illness which requires treatment.

Neuropsychological studies have shown that the cause of depressive disorders lies in a disturbance of the metabolism in the brain.

The balance between the neurotransmitters serotonin and noradrenalin is disturbed and they are either present in too low a concentration, or the signal transmission by these messenger substances is itself functionally disturbed.

Because of these proven physiological disturbances, depressive disorders are susceptible to medicinal therapy, with the therapy approach consisting in re-establishing the disturbed balance. This can be accomplished either by increasing the concentration of the neurotransmitters or by improving the neurological signal transmission.

In cases of slight depression or normal clinical pictures it is often sufficient to apply a therapy with a correspondingly selected antidepressant which has been adapted to the patient's condition. However, where the depressive disorder is resistant to treatment, it may be required to use various antidepressants to achieve an optimum therapeutic success for the patient.

On the other hand, it is precisely on account of the combination of different antidepressants with other psychotropic active agents that, frequently, severe undesired drug effects (UDEs) occur which are produced by interaction. For that reason it is indispensable not only that the active agents are accurately dosed, but also that intake schedules are strictly adhered to.

A further problem in the therapy consists in ensuring that the prescribed medicaments are in fact taken since the mental condition of the patients is often so bad that they forget to take them, or the patients often feel healthy and according to their own judgment do not require their medicaments, or because the patients distrust the medicaments and refuse to take them, for example by accumulating tablets in the mouth and spitting them out.

Common dosage forms for administering active agents in a therapy are tablets and capsules.

Tablets or capsules are easy to take, but they make it more difficult to check whether they have actually been taken, and the active agents, when absorbed via the gastrointestinal tract, are subject to the “first pass effect”, thus necessitating high initial active agent concentrations in the tablet or capsule. However, particularly in light of the previously mentioned range of UDEs produced by the combinations of active agents, dosages of active agents should be kept as low as possible to exclude serious side effects as far as possible. In addition, the extent of the first pass effect is strongly dependent on various factors, for example the quantity of the stomach content and thereby the “dilution” of the active agents, with the consequence that depending on the point in time of intake and on the metabolism conditions, highly differing plasma concentrations of the highly potent active agents are achieved. As a consequence, there are fluctuations both in terms of efficacy and side effects.

Furthermore, it is known to use buccal or sublingual tablets that release the active agent in the oral cavity, so that it can be absorbed directly via the oral mucosa.

The disadvantage of such tablets is an often unpleasant mouthfeel and, on account of the compact form, an only slow disintegration of the tablets, and, as a consequence thereof, a slow release of the active agents.

The task underlying the invention was therefore to provide pharmaceutical preparations by means of which a combination therapy for treating depressive disorders can be carried out in a simple and safe manner, and which enable avoidance or alleviation of the above-mentioned disadvantages. A further task underlying the invention was to indicate methods for the medicinal combination therapy of depressive disorders.

The improved therapy success of an active agent combination in the treatment of depression is due to the fact that different active agents are effective via different mechanisms, whereby, in addition, it is frequently possible to reduce the dose—and thereby the UDEs—of the individual active agent.

Moreover, by means of a combination therapy, it is also possible to treat different symptoms of depression or mental disorders accompanying the depression that are not covered by one active agent alone.

As previously mentioned, it has been found, however, that in patients treated with a combination therapy the frequency of UDEs, especially of severe UDEs such as grand mal attacks and suicides, is highly increased. For that reason, it is not only indispensable to dose the active agents accurately, but also to strictly adhere to the intake schedule.

As a compromise between checking the intake of the medication, which is easiest in the case of injections or infusions, and finding a practicable solution, orally administrable pharmaceutical forms represent a suitable means.

### SUMMARY OF THE INVENTION

It has been found that these tasks are solved by means of sheet-like pharmaceutical preparations in the form of wafer-like dosage forms, referred to as “wafers”, wherein the wafers, which are based on hydrophilic polymers, quickly disintegrate on contact with moisture and immediately release at least one active agent of an active agent combination which is, at least partially, transmucosally absorbed, and wherein at least one of the active agents of said active agent combination is selected from the antidepressant group of drugs.

It has furthermore been found that the use of an active agent combination comprising at least one antidepressant and a further psychotropic active agent is suitable for the production of a wafer for treating depressions.

Even where active agents are combined, a consistent intake and good compliance of the medicament are a prerequisite to ensuring optimal efficacy.

The administration of these active agent combinations in sheet-like dosage forms (wafers) not only enables easy intake but also the exact adaptation of the active agent components to each another, so that false dosages because intake has been forgotten or because of double intake of only one active agent, and thereby insufficient therapy of the depression, do not occur.

By means of the orally applied dosage form in the form of a wafer made of a quickly disintegrating hydrophilic polymer, intake of the medicaments by the patient is guaranteed since the wafer immediately disintegrates in the mouth.

In addition, the dosage form of the invention already contains an active agent combination adapted to the therapy, so that the administration of the medicaments needs to be supervised only once.

For patients who are themselves responsible for their medication, the wafer comprising an active agent combination considerably facilitates intake, so that the necessary consistent intake according to an intake schedule is guaranteed. Both compliance and therapy success are improved, and the risk of false applications is minimised.

The, at least partial, absorption of the active agents via the oral mucosa, as compared to other peroral dosage forms, affords the additional advantages that also patients having difficulty swallowing or patients refusing to take tablets can be administered medicaments via the oral route.

In addition, as a result of the parenteral administration, i.e. the direct absorption of the active agent via the mucous membrane, the active agents are not subject to the first-pass effect. Since in this way no active agent is metabolised before it reaches its site of action, the initial dose can be kept as low as possible. Furthermore, fluctuations in active agent concentration due to incomplete or delayed absorption, and delayed onset of action depending on the previously. ingested quantity of food are suppressed or minimised. Hence, the patient can be stabilised more reliably, and the necessity of taking the medicament on an empty stomach, for example, can be dropped.

Furthermore, a single active agent combination may contain active agents with different mechanisms of action having a synergistic effect, so that as a result of the different physiological activity and of the treatment of different symptoms of the depression, lower amounts of the active agents can be dosed than would be the case with single-component compositions.

In this connection, combinations of different antidepressants, as well as combinations of one or more antidepressants with anxiolytics, tranquilisers, nootropics, neuroleptics, sympathomimetics having psychoanaleptic effect, antiarrhythmics, sedatives and/or benzodiazepines may be utilised.

Since the active agent combinations in part exhibit new and severe UDEs, the number of active agents should be kept as low as possible. The wafers may therefore contain up to five, preferably up to three, and more preferably two active agents, with at least one of these active agents being an antidepressant.

By varying the ratios of the active agents to each other, it is, in addition, possible to adapt the dosages to the respective needs. Thus, depending on the symptoms of the depression, it is possible, for example, to administer a low-potency antidepressant or a low dose of antidepressant whereas to control intense unrest a corresponding sedative or benzodiazepine is administered.

It is thus possible to combine active agents in a suitable ratio to each other according to requirements, so that the treatment can be focused on different symptoms of the depression.

If the patient's mental condition changes, he or she can be easily stabilised on a dosage form with an altered active agent combination.

Because of the simple and low-cost manufacture of the wafers, it is possible to provide a large number of medicaments with different active agent concentrations.

Thus, if the wafer is made up of a laminate, it is possible, for example, to alter only the layer thickness of an active agent-containing layer, or to alter the concentration of the active agent.

On the other hand, medicaments can be produced which have different active agent contents but the same active agent ratio, simply by means of cutting the surface of the dosage form to different sizes.

### DETAILED DESCRIPTION OF THE PRESENT INVENTION

According to a further embodiment, one of the active agents may be effective immediately whereas a second active agent, for example an active agent with a shorter half-life, exhibits a retardation effect. Due to the retarded, continuous release, the plasma concentrations of the active agents remain constant over a prolonged period of time.

Furthermore, because of their flat shape the wafers of the invention, which contain active agent combinations, can be carried along easily, for example in a wallet, and are available at once, even when travelling. They are easy to take and they take effect quickly, both in regular therapy and in the case of suddenly occurring attacks, for example panic attacks, anxiety attacks and the like.

Water-soluble or swellable polymers that are suitable as a base polymer for the hydrophilic water-soluble and/or swellable polymer film are polymers of the group comprising dextran, polysaccharides, inclusive of starch and starch derivatives, cellulose derivatives, such as carboxymethyl cellulose, ethyl cellulose or propyl cellulose, hydroxypropylmethyl cellulose, hydroxypropyl cellulose, sodium carboxymethyl cellulose (e.g. WALOCEL®), methyl cellulose, hydroxyethyl cellulose and hydroxypropylethyl cellulose, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of these polymers. As an alternative, the polymer film may be made of a polyvinyl alcohol-polyethylene glycol graft copolymer.

The proportion of polymer in a dosage form according to the invention is preferably 5 to 95%-wt., more preferably 15 to 75%-wt., relative to the dry mass of the dosage form.

Suitable antidepressants for use in a combination wafer comprise the tricyclic, tetracyclic and noncyclic antidepressants, as well as the general active agent groups of the phenothiazines, azaphenothiazines, thioxanthenes, butyrophenones, diphenylbutyl piperidines, iminodibenzyl derivatives, iminostilbene derivatives, dibenzocycloheptadiene derivatives, dibenzodiazepine derivatives, dibenzoxepine derivatives, benzodiazepines, indole derivatives, phenylethylamine derivatives and hypericin derivatives, as well as pharmaceutically acceptable salts or derivatives of these compounds. Furthermore, selective serotonin reuptake inhibitors (SSRIs) may likewise be used as antidepressants.

Examples of selected active agents from these groups of antidepressants are amitryptyline, amitryptyline oxide, buspirone, citalopram, clomipramine, desipramine, dibenzepin, dosulepin, doxepin, fluoxetine, fluvoxamine, imipramine, lofepramine, maprotiline, mianserin, mirtazapine, moclobemide, nefazodone, nortriptyline, opipramol, oxitriptan, paroxetine, reboxetine, sertraline, tranylcypromine, trimipramine, venlafaxine and viloxazine, as well as pharmaceutically acceptable salts of these active agents.

Suitable for the therapy of depression are furthermore the previously mentioned groups of active agents, i.e. anxiolytics, tranquilisers, nootropics, neuroleptics, sympathomimetics having psychoanaleptic effect, antiarrhythmics, sedatives and/or benzodiazepines, as well as the active agents selected from these groups, such as caffeine, meprobamate, reserpine, prolintane, hydroxyzine, lorazepam, chlorpromazine, clozapine, perphenazine, risperidone, sulpiride, meclofenoxate, nicergoline, piracetam, pyritinol, fenetylline and methylphenidate, as well as pharmaceutically acceptable salts of these active agents.

In this regard, the active agent combinations of the invention contain at least one antidepressant and optionally one or more further active agents of the other groups of active agents.

In one particular embodiment, at least one active agent is a selective serotonin reuptake inhibitor (SSRI), such as fluoxetine or paroxetine.

In one embodiment according to the invention, the active agent combination consists of mirtazapine and lorazepam or pharmacologically acceptable salts of these substances, the ratio of mirtazapine to lorazepam being 95%-80% to 5%-20%, preferably 88% to 12%.

In another preferred embodiment, the active agent combination comprises three active agents, namely mirtazapine, lorazepam and risperidone, or pharmacologically acceptable salts thereof, the ratio of mirtazapine to lorazepam to risperidone preferably being 75% to 10% to 15%, other compositions also being possible.

Another preferred active agent combination consists of the anxiolytic buspirone and tranylcypromine, reboxetine or mirtazapine.

In one particular embodiment, the active agent combination comprises the anxiolytic buspirone and lorazepam, with the ratio of buspirone to lorazepam being 95%-80% to 5%-20%, preferably 88% to 12%.

The ratio of the active agents to each other can be varied freely and depends on the potency of the active agent, on the UDEs occurring, and on the severity of the symptoms to be treated.

To improve the physicochemical properties, for example reduce the brittleness or embrittlement, humectants, such as glycerine, propylene glycol, sorbitol, mannitol, polyethylene glycol, polyglycerol ester and the like, may be added to the film.

In a further embodiment, antioxidants, for example vitamin C (ascorbic acid), ascorbyl palmitate, vitamin E (tocopherol acetate), hydroxybenzoic acid derivatives, may be added to the wafer, in order to stabilise the film and the active agents. Furthermore, acidic and basic ion exchangers may be used as stabilisers.

In further embodiments, further ingredients such as dyes, pigments, taste flavourings, natural and/or synthetic flavouring substances, sweeteners, buffering systems, may be added to the film. In particular, the taste flavourings and flavouring substances can cover the often bad inherent taste or smell of the active agents and/or give the dosage form a pleasant taste, so that the patient's readiness to take the medication is considerably improved. To mask the taste, it is also possible for the active agent(s) to be bound to an acidic or basic ion exchanger.

The addition of buffering systems on the one hand serves to stabilise the film and the active agents against outside influences and during storage. On the other hand, the pH of the dosage form can thereby be adjusted to a physiologically acceptable pH value, so that irritation of mucous membranes is avoided. By using a buffering system, it is also possible to improve the solubility of acidic or basic active agents in the matrix.

The dosage forms according to the invention are configured so as to be thin, for example in the form of a wafer. The thickness of the dosage form is preferably 0.1 to 5 mm, more preferably 0.5 to 1 mm. The lower limit for the thickness of the dosage form is about 50 μm. The surface area of the dosage form is between 0.09 cm² and 12 cm², preferably between 1 cm² and 8 cm², and more preferably between 3 cm² and 6 cm².

In a further embodiment, the wafers of the present invention contain a disintegrant or a wicking agent, for example a bicarbonate-acid mixture or an aerosil, being activated by contact with a liquid and accelerating the disintegration of the wafer after application thereof, and thereby also accelerating the release of active agent.

In another preferred embodiment, the wafer is present as a foam so that the release of active agent takes place even more rapidly because of the enlarged surface. In this embodiment, the cavities of the foam may contain one or more of the active agents in liquid form.

To improve the absorption of the active agents via the mucous membrane, permeation enhancers, such as substances from the groups of the fatty alcohols, fatty acids, polyoxyethylene fatty alcohol ethers, polyoxyethylene fatty acid esters, fatty alcohol esters and fatty acid esters, particularly sorbitan monolaurate or esters of long-chain fatty acids with methyl, ethyl or isopropyl alcohol, or esters of fatty alcohols with acetic acid or lactic acid, or substances such as DMSO (dimethyl sulfoxide) and oleic acid diethanolamine may likewise be incorporated in the film. The constituent amount of these substances is 0.1%-wt. to 25%-wt., preferably 1 to 10%-wt., in each case relative to the total weight of the active agent matrix.

Furthermore, the composition of the wafer may contain compounds that retard the release of active agent (e.g., microencapsulation).

In a further embodiment, the wafer has mucoadhesive properties, so that it adheres to the mucous membrane until it is completely dissolved. This embodiment, in addition, makes it easier to supervise the administration of the medicaments since the wafer, which adheres to the mucous membrane, cannot be spit out.

In another preferred embodiment, at least one of the active agents is bound to an ion exchanger, so that the hydrophilic polymer disintegrates quickly in the oral cavity, whereas the release of active substance is retarded or occurs when the pH has changed, e.g. in the gastrointestinal tract. In this way, active agents having a different mechanism of action and absorption can be administered in one dosage form, that is, at least one of the released active agents is absorbed at the site of application, for example via the mucous membrane, and the other active agent is transported farther and absorbed at another site.

The wafer may also be made up as a laminate with different layers, with the active agents being contained in discrete layers which are spatially separated from each other and differ from each other in terms of their composition. In this way, the active agents can be released at different sites of action, but also with retardation if the disintegration times of the various layers of the wafer differ from each other.

Likewise, the active agents may be arranged within layers that disintegrate at different rates, so that the preparation as a whole shows a retardation effect.

In a further embodiment, only one of the outer layers may be mucoadhesive, to promote the adherence of the dosage form on the mucous membrane and to facilitate the active agent absorption via the mucous membrane by establishing direct contact.

The disintegration of the inventive dosage form in an aqueous medium preferably takes place in the range from 1 second to 5 minutes, more preferably in a range from 5 seconds to 1 minute, and most preferably in the range from 10 seconds to 30 seconds.

The dosage forms according to the invention are advantageously suitable for administering medicaments in the oral cavity or for rectal, vaginal or intranasal administration. They can be used both in human medicine and in veterinary medicine.

The present invention furthermore relates to the use of an active substance combination of the invention for the production of an oral dosage form for treating depressive disorders, said dosage form preferably being formulated as a wafer.

Furthermore, the present invention relates to a method for the therapeutic treatment of a person suffering from depression, wherein the administration of an above-described active agent combination of antidepressants is carried out by means of an orally applicable dosage form with at least partial transmucosal absorption of at least one active agent.

Finally, the present invention also relates to a method for the production of a sheet-like dosage form, comprising the following steps:
preparing a solution containing at least one polymer and at least two active agents from the antidepressant group of drugs;
spread-coating the solution on a coating substrate, and
solidifying the spread-coated solution by drying and withdrawing the solvent.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A sheet-like pharmaceutical preparation (dosage form) that quickly disintegrates on contact with moisture, based on hydrophilic polymers, for the release of at least one active agent in a body orifice or body cavity, wherein the dosage form contains an active agent combination for treating depression, with at least one of the active agents of the active agent combination being selected from the group consisting of antidepressant drugs.

2. The pharmaceutical preparation according to claim 1, wherein further active agents contained in the active agent combination are selected from the group consisting of antidepressants, anxiolytics, tranquilisers, nootropics, neuroleptics, sympathomimetics having psychoanaleptic effect, antiarrhythmics, sedatives and benzodiazepines.

3. The pharmaceutical preparation according to claim 1, wherein the active agent combination comprises 2 to 5 active agents.

4. The pharmaceutical preparation according to claim 1, wherein the active agents are selected from the group consisting of phenothiazines, azaphenothiazines, thioxanthenes, butyrophenones, diphenylbutyl piperidines, iminodibenzyl derivatives, iminostilbene derivatives, dibenzocycloheptadiene derivatives, dibenzodiazepine derivatives, dibenzoxepine derivatives, benzodiazepines, indole derivatives, phenylethylamine derivatives and hypericin derivatives, as well as pharmaceutically acceptable salts or derivatives of said active agents.

5. The pharmaceutical preparation according to claim 1, wherein the antidepressant is selected from the group consisting of tricyclic antidepressants, tetracyclic antidepressants and noncyclic antidepressants.

6. The pharmaceutical preparation according to claim 5, wherein the antidepressant is selected from the group consisting of selective serotonin reuptake inhibitors.

7. The pharmaceutical preparation according to claim 1, wherein the active agent of the antidepressant is selected from the group consisting of amitryptyline, amitryptyline oxide, buspirone, citalopram, clomipramine, desipramine, dibenzepin, dosulepin, doxepin, fluoxetine, fluvoxamine, imipramine, lofepramine, maprotiline, mianserin, mirtazapine, moclobemide, nefazodone, nortriptyline, opipramol, oxitriptan, paroxetine, reboxetine, sertraline, tranylcypromine, trimipramine, venlafaxine and viloxazine, as well as pharmaceutically acceptable salts of said active agents.

8. The pharmaceutical preparation according to claim 1, wherein further active agents of the active agent combination are selected from the group consisting of the antidepressants as well as caffeine, meprobamate, reserpine, prolintane, hydroxyzine, lorazepam, chlorpromazine, clozapine, perphenazine, risperidone, sulpiride, meclofenoxate, nicergoline, piracetam, pyritinol, fenetylline and methylphenidate, as well as pharmaceutically acceptable salts of said active agents.

9. The pharmaceutical preparation according to claim 1, wherein the active agent combination comprises mirtazapine and lorazepam or pharmacologically acceptable salts thereof.

10. The pharmaceutical preparation according to claim 1, wherein the active agent combination comprises mirtazapine, lorazepam and risperidone or pharmacologically acceptable salts thereof.

11. The pharmaceutical preparation according to claim 1, wherein one active agent of the active agent combination is selected from the group consisting of tranylcypromine, reboxetine and mirtazapine, and that the second active agent is buspirone.

12. The pharmaceutical preparation according to claim 1, wherein one active agent of the active agent combination is lorazepam, and the second active agent is buspirone.

13. The pharmaceutical preparation according to claim 1, wherein the hydrophilic polymer is selected from the group consisting of dextran, polysaccharides, inclusive of starch and starch derivatives, cellulose derivatives, polyvinyl alcohols, polyethylene glycols, polyacrylic acids, polyacrylates, polyvinylpyrrolidones, alginates, pectins, gelatine, alginic acid, collagen, chitosan, arabinogalactan, galactomannan, agar-agar, agarose, carrageenan natural gums, tragacanth, highly dispersed silicon dioxide, bentonite, as well as derivatives of the aforementioned hydrophilic polymers or combinations of two or more of said polymers.

14. The pharmaceutical preparation according to claim 1, wherein the polymer film comprises a polyvinyl alcohol-polyethylene glycol graft copolymer.

15. The pharmaceutical preparation according to claim 1, wherein the preparation further comprises a humectant selected from the group consisting of glycerine, propylene glycol, sorbitol, mannitol, polyethylene glycol and polyglycerol ester.

16. The pharmaceutical preparation according to claim 1, wherein the preparation further comprises an antioxidant selected from the group consisting of vitamin C (ascorbic acid), ascorbyl palmitate, vitamin E (tocopherol acetate) and hydroxybenzoic acid derivatives.

17. The pharmaceutical preparation according to claim 1, wherein the active agent of the preparation is bound to an acidic or basic ion exchanger for taste masking.

18. The pharmaceutical preparation according to claim 1, wherein the preparation further comprises at least one of dyes and pigments.

19. The pharmaceutical preparation according to claim 1, wherein the preparation contains natural and/or synthetic flavouring substances.

20. The pharmaceutical preparation according to claim 1, wherein the preparation further comprises a disintegrant or a wicking agent.

21. The pharmaceutical preparation according to claim 1, wherein said preparation further comprises a buffer system for adjusting the pH value of the preparation.

22. The pharmaceutical preparation according to claim 1, wherein the hydrophilic polymer disintegrates within less than 5 minutes after application in the oral cavity of a user of the pharmaceutical preparation.

23. The pharmaceutical preparation according to claim 1, wherein the hydrophilic polymer disintegrates quickly in the oral cavity whereas the active agent remains. bound to an ion exchanger which releases said active agent only in the gastrointestinal tract of a user of the pharmaceutical preparation.

24. The pharmaceutical preparation according to claim 1, wherein the active agents are contained in discrete layers which are spatially separated from each another and which differ from each other in terms of their respective composition.

25. The pharmaceutical preparation according to claim 1, wherein the preparation is present as a foam having cavities and at least one of the active agents is present in liquid form within the cavities of said foam.

26. Use of an active agent combination as according to claim 1, for the production of an orally administrable pharmaceutical product in the form of a wafer for treating depressions.

27. A method for the therapeutic treatment of a person suffering from depression, comprising the step of orally administering an active agent combination to the person, wherein said active agent combination quickly disintegrates on contact with moisture, based on hydrophilic polymers, for the release of at least one active agent in a body orifice or body cavity of a user of the person, and contains an active agent combination for treating depression, with at least one of the active agents of the active agent combination being selected from the group consisting of antidepressant drugs.

28. The method according to claim 27, wherein the step of administering the active agent combination comprises quickly disintegrating wafer a that contains said combination.

29. The pharmaceutical preparation according to claim 3, wherein the active agent combination comprises 2 active agents.

30. The pharmaceutical preparation according to claim 13, wherein said cellulose derivatives are selected from the group consisting of carboxymethyl cellulose, ethyl cellulose, propyl cellulose, hydroxypropylmethyl cellulose, hydroxypropyl cellulose, sodium carboxymethyl cellulose, methyl cellulose, hydroxyethyl cellulose and hydroxypropylethyi cellulose.

31. The pharmaceutical preparation according to claim 22, wherein the hydrophilic polymer disintegrates within less than 3 minutes after application in the oral cavity of a user of the pharmaceutical preparation.

32. The pharmaceutical preparation according to claim 31, wherein the hydrophilic polymer disintegrates within less than 1 minute after application in the oral cavity.

33. The pharmaceutical preparation according to claim 32, wherein the hydrophilic polymer disintegrates within less than 30 seconds after application in the oral cavity.
